(19) United States
(12) Patent Application Publication
(10) Pub. No.: US 2026/0315282 A1
(43) Pub. Date: Oct. 15, 2026

(54) EARPLUG ATTACHMENT COMPONENT

(71) Applicant: Morgan Wynell McCLARY, Spring Hill, TN (US)

(72) Inventor: Morgan Wynell McCLARY, Spring Hill, TN (US)

(21) Appl. No.: 19/466,557

(22) Filed: Feb. 2, 2026

Related U.S. Application Data
(60) Provisional application No. 63/753,342, filed on Feb. 3, 2025.

(51) Int. Cl. A61F 11/12

(52) U.S. Cl. CPC A61F 11/12

(57) ABSTRACT

Present disclosure describes an earplug attachment component. The earplug attachment component comprises at least one unitary body extending along a longitudinal axis. The unitary body comprises a mounting portion present one end. The mounting portion comprises a through-hole configured to receive a fastening element. The unitary body further comprises a lower body portion present at another end. The lower body portion comprises a flared geometry having a cavity configured to receive at least one object comprising an earplug. The unitary body further comprises a tapered transition portion extending from the mounting portion to the lower body portion. The tapered transition portion gradually increases in cross-sectional dimension from the mounting portion toward the lower body portion.

### TECHNICAL FIELD

The present disclosure relates generally to earplug, and more specifically to an attachment component for earplug.

### BACKGROUND

Current hearing protection accessories often rely on a basic silicone cord to link a pair of earplugs. While these tethers are designed to prevent the loss of individual plugs, they are frequently made from low-friction materials that offer little grip against skin or clothing. This fundamental design flaw means the cord rarely stays centered, often shifting as the wearer moves, which can be both distracting and uncomfortable during physical activity or work.

The functional failure of these standard attachments extends beyond mere annoyance to a genuine risk of equipment loss. Because silicone is lightweight and lacks structural stability, the cord can easily slide off the back of a user's neck without them even noticing. For professionals in industrial settings or musicians on stage, this lack of security means that high-quality, often expensive earplugs can be permanently lost the moment they are removed from the ear canal.

Beyond the physical mechanics, there is a distinct aesthetic disconnect in the current market. Most silicone cords are utilitarian and sterile in appearance, lacking any sense of personal style or modern design. This “medicalized” look often clashes with the wearer's attire, making the safety equipment feel like an eyesore rather than a seamless part of their daily kit. In environments where image matters, these bulky, unattractive strings are often the first thing users choose to leave behind.

This lack of visual appeal creates a significant psychological barrier to consistent safety compliance. If a user feels that their hearing protection is unfashionable or cumbersome, they are far less likely to wear it in moderately loud environments where protection is still necessary. The “fashion tax” of wearing a standard cord effectively disincentivizes the very habit that is meant to prevent long-term auditory damage, leaving users vulnerable to noise-induced hearing loss.

Ultimately, there is a clear demand for a more sophisticated solution that bridges the gap between security and style. An ideal attachment would need to provide a reliable, “stay-put” grip while offering an aesthetic that feels like a conscious accessory choice rather than a mandatory safety tether. By addressing these functional and visual drawbacks, a redesigned attachment could transform hearing protection from a chore into a desirable, everyday habit.

The information disclosed in this background section is only for enhancement of understanding of the general background as identified in the disclosure and should not be taken as an acknowledgement or any form of suggestion that this information forms the prior art already known to a person skilled in the art.

### SUMMARY

The present disclosure overcomes one or more shortcomings of the prior art and provides additional advantages discussed throughout the present disclosure. Other aspects and aspects of the disclosure are described in detail herein and are considered a part of the claimed disclosure.

This summary is provided to introduce a selection of concepts, in a simple manner, which is further described in the detailed description of the disclosure. This summary is neither intended to identify key or essential inventive concepts of the subject matter nor to determine the scope of the disclosure.

In an aspect of the present disclosure, an attachment component. The attachment component comprises at least one unitary body extending along a longitudinal axis. The at least one unitary body comprises a mounting portion present one end. The mounting portion comprises a through-hole configured to receive a fastening element. The unitary body further comprises a lower body portion present at another end. The lower body portion comprises a flared geometry having a cavity configured to receive at least one object comprising an earplug. The unitary body further comprises a tapered transition portion extending from the mounting portion to the lower body portion. The tapered transition portion gradually increases in cross-sectional dimension from the mounting portion toward the lower body portion.

In an aspect of the present disclosure, the at least one unitary body is rotationally symmetric about the longitudinal axis.

In an aspect of the present disclosure, the cavity comprises a circular or a concave cavity.

In an aspect of the present disclosure, the cavity is configured to stretch and accommodate the earplug.

In an aspect of the present disclosure, the unitary body is made up of stretchable material and tear resistant material.

In an aspect of the present disclosure, the attachment component is formed as a single moulded component moulded by pouring stretchable and tear resistant material.

In an aspect of the present disclosure, the fastening element comprises clasp comprising trigger clasp, hook-and-latch fastener, and jewelry clasp.

In an aspect of the present disclosure, the fastening element comprises string, jump ring, or an accessory worn by a user.

In an aspect of the present disclosure, the attachment component is coupled with earring attachments.

In an aspect of the present disclosure, the attachment component is coupled with clasping necklace, glasses attachments, or an accessory worn by a user.

The foregoing summary is illustrative only and is not intended to be in any way limiting. In addition to the illustrative aspects, and features described above, further aspects, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary aspects and, together with the description, serve to explain the disclosed principles. Some aspects of system and/or methods in accordance with aspects of the present subject matter are now described, by way of example only, and with reference to the accompanying figures, in which:

FIG. 1(a) shows an environment 100 diagram for implementing the earplug attachment component, in accordance with some embodiments of the present disclosure;

FIG. 1(b) shows the earplug attachment component at various angles, including a side view 1B and back view 1C, in accordance with some embodiments of the present disclosure.

FIG. 2 illustrates the attachment component 2A connected to a high-fidelity earplug 2B, 2C, in accordance with some embodiments of the present disclosure.

FIG. 3 illustrates the attachment component 3A connected to a loop-style high-fidelity earplug 3B, 3C, in accordance with some embodiments of the present disclosure.

FIG. 4 displays the attachment component 4A in use with an earring attachment 4B and an earplug 4C within a human ear 4D, in accordance with some embodiments of the present disclosure; and

FIG. 5 shows the attachment component 5A connected to a magnetic clasping necklace (5B, 5D), in accordance with some embodiments of the present disclosure;

It should be appreciated by those skilled in the art that any block diagrams herein represent conceptual views of the illustrative systems embodying the principles of the present subject matter.

### DETAILED DESCRIPTION

In the present document, the word “exemplary” is used herein to mean “serving as an example, instance, or illustration.” Any aspect or implementation of the present subject matter described herein as “exemplary” is not necessarily to be construed as preferred or advantageous over other aspects.

While the disclosure is susceptible to various modifications and alternative forms, specific aspects thereof have been shown by way of example in the drawings and will be described in detail below. It should be understood, however, that these aspects are not intended to limit the disclosure to the particular form disclosed, but on the contrary, the disclosure is to cover all modifications, equivalents, and alternatives falling within the spirit and the scope of the disclosure.

The terms “comprise(s)”, “comprising”, “include(s)”, or any other variations thereof, are intended to cover a non-exclusive inclusion, such that a setup, device, apparatus, system, or method that comprises a list of components or steps does not include only those components or steps but may include other components or steps not expressly listed or inherent to such setup or device or apparatus or system or method. In other words, one or more elements in a device or system or apparatus proceeded by “comprises . . . a” does not, without more constraints, preclude the existence of other elements or additional elements in the system.

The expressions like “at least one” and “one or more” may be used interchangeably or in combination throughout the description.

In the following detailed description of the aspects of the disclosure, reference is made to the accompanying drawings that form a part hereof, and in which are shown by way of illustration of specific aspects in which the disclosure may be practiced. These aspects are described in sufficient detail to enable those skilled in the art to practice the disclosure, and it is to be understood that other aspects may be utilized and that changes may be made without departing from the scope of the present disclosure. The following description is, therefore, not to be taken in a limiting sense. In the following description, well known functions or constructions are not described in detail since they would obscure the description with unnecessary detail.

The terminologies “earplug attachment component” and “attachment component 103” have same meaning and are interchangeably used throughout the specification.

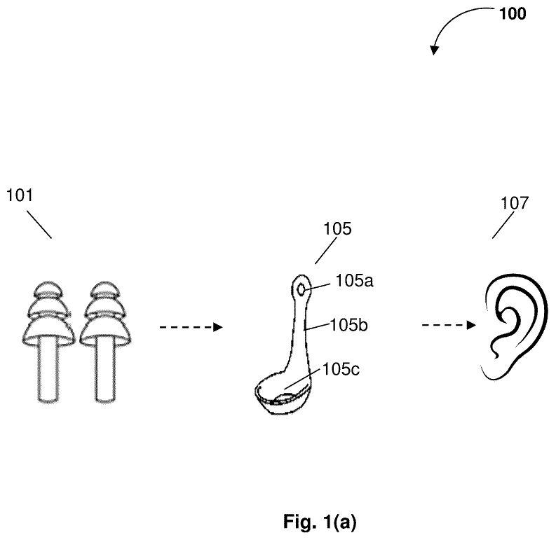

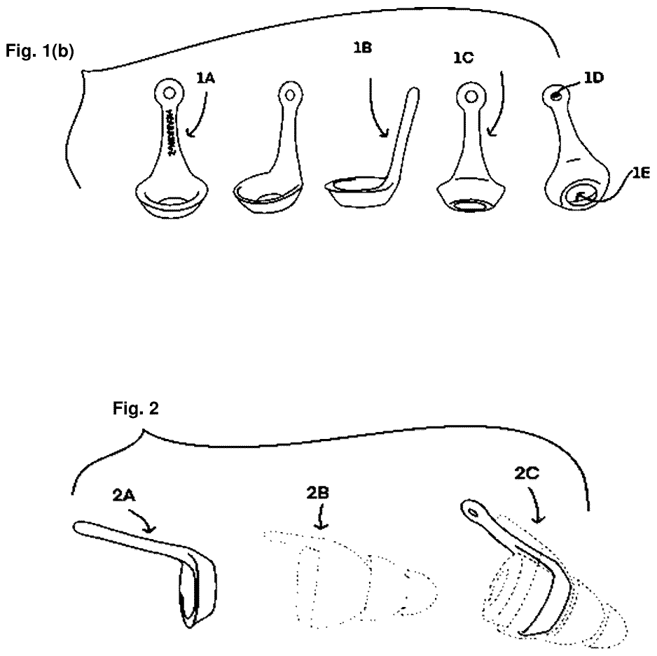

FIG. 1(a) shows an environment 100 diagram for implementing the earplug attachment component, in accordance with some embodiments of the present disclosure.

In an aspect of the present disclosure, the environment 100 may comprise an earplug 101 and an earplug attachment component 103. The earplug attachment component 103 may be configured to accommodate the earplug 101 and the earplug attachment component 103 may be then worn by the user's ear 107.

The earplug attachment component 103 may comprise a unitary body extending along a longitudinal axis. The unitary body may comprise a mounting portion 103a present one end. The mounting portion 103a may comprise a through-hole configured to receive a fastening element. The fastening element may comprise, but not limited to, clasp comprising trigger clasp, hook-and-latch fastener, and jewelry clasp. In another aspect of the present disclosure, the fastening element comprises string, jump ring, or an accessory worn by a user.

The unitary body may further comprise a lower body portion 103c present at another end. The lower body portion 103c may comprise a flared geometry having a cavity configured to receive at least one object comprising an earplug. However, the present disclosure is not limited to earplug of a specific shape and size and the cavity of the lower body portion 103c may be configured to stretch and accommodate earplug of any shape and size.

In one non-limiting aspect of the present disclosure, the cavity of the lower body portion 103c may be configured to accommodate any other hearing aid/device to be worn by the user to facilitate different advantages of the present disclosure.

In an aspect, the unitary body may further comprise a tapered transition portion 103b extending from the mounting portion 103a to the lower body portion 103c. In one non-limiting aspect, the tapered transition portion 103b may gradually increases in cross-sectional dimension from the mounting portion toward the lower body portion. In another non-limiting aspect, the tapered transition portion 103b may have a constant cross section throughout. However, the middle portion of the unitary body is not limited to above examples and other geometrical shapes known to a person is well within the scope of the present disclosure.

In an aspect, the unitary body is made up of stretchable material and tear resistant material. The attachment component is formed as a single moulded component moulded by pouring stretchable and tear resistant material.

Thus, the earplug attachment component 103 facilitates reduction in the likelihood of losing earplugs. Because many non-custom earplugs tend to eject or slide out of the ear during use, this earplug attachment component 103 ensures they remain attached to the user rather than falling onto the ground and getting dirty or being lost entirely.

FIG. 1(b) shows the earplug attachment component at various angles, including a side view 1B and back view 1C, in accordance with some embodiments of the present disclosure.

The earplug attachment component 1A is a specialized connector designed to integrate earplugs with decorative or functional accessories. In its primary view, the earplug attachment component appears as a compact, modular piece that serves as the central hub for attachment. The side view 1B highlights its slim, ergonomic profile, ensuring that it sits comfortably along the ear without adding bulk. Meanwhile, the back view 1C reveals the stabilizing surface that helps secure the component in place, preventing unwanted movement during use. Together, these perspectives emphasize the balance between functionality and discreet design, making the component suitable for both everyday wear and specialized acoustic applications.

At the structural level, the top connection part or mounting portion 1D is engineered to allow jewelry or accessories to be easily attached, enabling personalization and aesthetic enhancement. This feature transforms a purely functional earplug into a customizable accessory. The bottom connection or the lower body portion 1E is the critical interface that links directly to the earplugs, ensuring a secure and reliable fit. By combining these two connection points, earplug attachment component achieves dual functionality: maintaining the protective role of earplugs while offering a stylish, modular platform for adornment. This dual-purpose design reflects a thoughtful blend of acoustic utility and personal expression.

FIG. 2 illustrates the attachment component 2A connected to a high-fidelity earplug 2B, 2C, in accordance with some embodiments of the present disclosure.

The earplug attachment component 2A is a modular connector designed to integrate seamlessly with high-fidelity earplugs. Its structure allows for secure attachment while maintaining a discreet and ergonomic profile. For example, the Eargasm High Fidelity Earplug 2B, known for its acoustic precision and comfort, serves as the base hearing protection device. When viewed individually, each component fulfils a distinct role: the earplug attachment component provides the customizable interface for accessories, while Eargasm delivers the core function of sound attenuation without compromising clarity.

When combined, the high-fidelity earplug attachment component connected to an Eargasm earplug 2C demonstrates the synergy between utility and personalization. The attachment locks firmly onto the earplug, ensuring stability during use, while still allowing the wearer to add jewelry or decorative elements through the earplug attachment component top connection feature. This integration preserves the acoustic performance of the Eargasm earplug while enhancing its aesthetic versatility, effectively transforming a purely functional hearing protection device into a customizable accessory that balances style with high-fidelity sound protection.

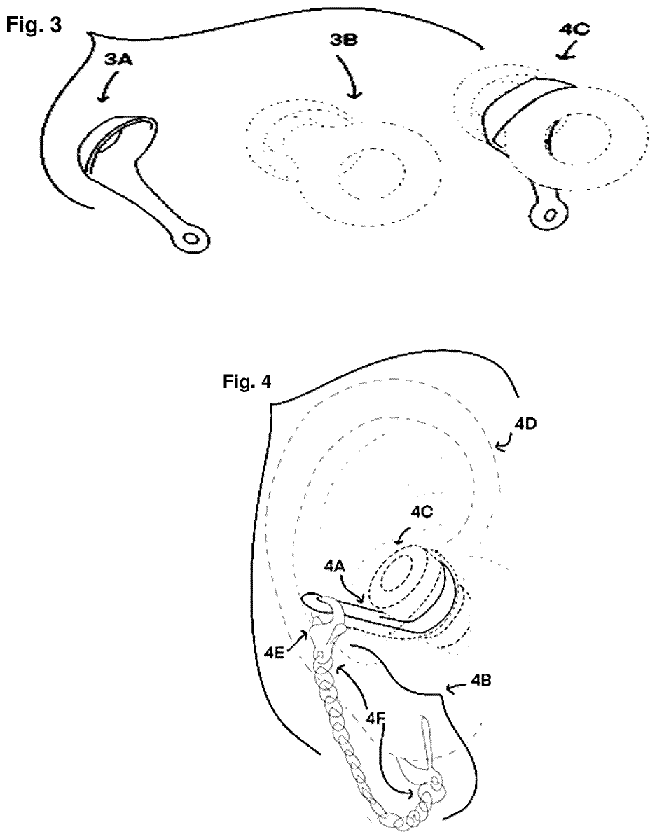

FIG. 3 illustrates the attachment component 3A connected to a loop-style high-fidelity earplug 3B, 3C, in accordance with some embodiments of the present disclosure.

The earplug attachment component 3A is designed as a modular connector that enables personalization and accessory integration with earplugs. Its compact form ensures that it can be paired with different earplug types without interfering with their acoustic performance. The Loop High Fidelity Earplug 3B, known for its circular design and advanced sound filtering, provides clear sound reduction while maintaining audio fidelity. Each component serves a distinct purpose: earplug attachment component offers the customizable interface for jewelry or decorative attachments, while Loop delivers the functional benefit of high-quality hearing protection.

When combined, the earplug attachment component connected to a Loop High Fidelity Earplug 3C demonstrates a seamless integration of style and utility. The attachment locks securely onto the Loop earplug, preserving its ergonomic fit and acoustic precision while adding the option for aesthetic enhancement. This pairing transforms the earplug from a purely functional hearing protection device into a versatile accessory that balances fashion with performance, allowing users to enjoy both high-fidelity sound attenuation and personalized expression in a single integrated system.

FIG. 4 displays the attachment component 4A in use with an earring attachment 4B and an earplug 4C within a human ear 4D, in accordance with some embodiments of the present disclosure.

The earplug attachment component 4A works in tandem with the Earring Attachment 4B to provide a modular system that combines hearing protection with personal adornment. When connected to the Eargasm High Fidelity Earplug 4C, the assembly maintains the earplug's acoustic performance while enabling decorative customization through the earring interface. Positioned on the human ear 4D, this integrated design demonstrates how earplug attachment component transforms a functional hearing protection device into a dual-purpose accessory, offering both high-fidelity sound attenuation and aesthetic enhancement in a single, ergonomic configuration

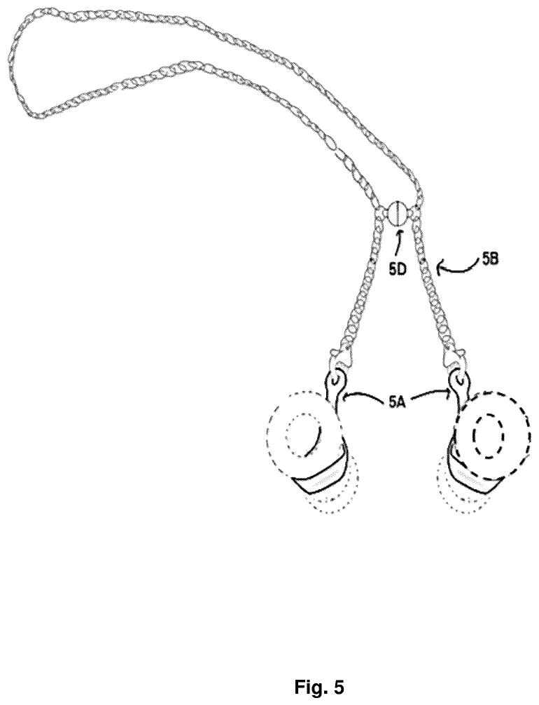

FIG. 5 shows the attachment component 5A connected to a magnetic clasping necklace (5B, 5D), in accordance with some embodiments of the present disclosure

The earplug attachment component 5A is designed as a modular connector that enables integration between hearing protection devices and decorative accessories. The Magnetic Clasping Necklace Attachment 5B extends this functionality by allowing jewelry, specifically necklaces, to be securely fastened to the earplug system. The Loop High Fidelity Earplug 5C serves as the acoustic foundation, providing advanced sound filtering and clarity while maintaining comfort. Each element plays a distinct role: earplug attachment component provides the interface, and the necklace attachment introduces aesthetic versatility, and Loop ensures high-quality hearing protection.

When combined, the Magnetic Clasp 5D becomes the critical mechanism that secures the necklace attachment to the earplug component, ensuring both stability and ease of use. This integration demonstrates how earplug attachment transforms a functional hearing protection device into a dual-purpose accessory system. The wearer benefits from the acoustic precision of Loop earplugs while enjoying the personalization of jewelry attachments, all held together by the magnetic clasp's reliable connection. The result is a seamless blend of utility and style, merging high-fidelity sound attenuation with customizable fashion in a single ergonomic design.

### USES

Method 1. Earplug Attachment Component 1A can be used with earring attachments (4B) and a high-fidelity earplug as shown in FIG. 4.
Method 2. Earplug Attachment Component 1A can be used with a Magnetic Clasping Necklace 5B attachment and high-fidelity earplugs as shown in FIG. 5.
Method 3. Earplug Attachment Component 1A can be used with the glasses attachments and high-fidelity earplugs.

Along with these methods there are multiple other combinations of attachments and earplugs that can be used with the Earplug Attachment Component 1A.

The terms “an aspect”, “aspect”, “aspects”, “the aspect”, “the aspects”, “one or more aspects”, “some aspects”, and “one aspect”, “other aspect”, “yet another aspect”, “non-limiting aspect” mean “one or more (but not all) aspects of the disclosure(s)” unless expressly specified otherwise.

The terms “including”, “comprising”, “having” and variations thereof mean “including but not limited to”, unless expressly specified otherwise.

The enumerated listing of items does not imply that any or all the items are mutually exclusive, unless expressly specified otherwise.

A description of an aspect with several components in communication with each other does not imply that all such components are required. On the contrary, a variety of optional components are described to illustrate the wide variety of possible aspects of the disclosed system.

Finally, the language used in the specification has been principally selected for readability and instructional purposes, and it may not have been selected to delineate or circumscribe the inventive subject matter. It is therefore intended that the scope of the disclosure be limited not by this detailed description. Accordingly, the aspects of the present disclosure are intended to be illustrative, but not limiting, of the scope of the disclosure.

### ADVANTAGES OF THE PRESENT DISCLOSURE

Exemplary embodiments discussed above may provide certain advantages. Though not required to practice aspects of the disclosure, these advantages may include those provided by the following features.

Prevention of Loss and Damage: The primary advantage is a significant reduction in the likelihood of losing earplugs. Because many non-custom earplugs tend to eject or slide out of the ear during use, this attachment component 103 ensures they remain attached to the user rather than falling onto the ground and getting dirty or being lost entirely.

Universal Compatibility: Unlike proprietary accessories, this attachment component 103 is designed to be a universal solution. Testing has confirmed that it matches for all notable high-fidelity earplug brands, including Eargasm, Loop, HearProtek, and like, and it is projected to work with many other brands.

Enhanced Durability: The attachment component 103 is manufactured from a strong, stretchy, and tear-resistant material. This allows it to be stretched over various earplug bodies without compromising its structural integrity.

Aesthetic Appeal and Fashion: One of the main drawbacks of existing silicone cords is that they are not considered fashionable. The present disclosure incentivizes the use of hearing protection by allowing earplugs to be integrated into jewelry and accessories, making them more stylish and accessible for the wearer.

Ease of Use and Storage: The present disclosure addresses the “annoyance” many consumers face when frequently inserting, removing, and storing their earplugs. By keeping the earplugs attached to an earring (FIG. 4), necklace (FIG. 5), or glasses, the user can manage them effortlessly throughout the day.

Superior Security compared to Silicone Cords: Standard silicone cords are prone to sliding around and can fall off the user's neck. The attachment component 103 provides a more secure tethering method via claw clasps, jump rings, or magnetic clasping necklaces, ensuring the earplugs stay on the person.

What is claimed is:

1. An attachment component comprising:
at least one unitary body extending along a longitudinal axis,
wherein the unitary body comprises:
a mounting portion present one end, wherein the mounting portion comprises a through-hole configured to receive a fastening element;
a lower body portion present at another end, wherein the lower body portion comprises a flared geometry having a cavity configured to receive at least one object comprising an earplug, and
a tapered transition portion extending from the mounting portion to the lower body portion;
wherein the tapered transition portion gradually increases in cross-sectional dimension from the mounting portion toward the lower body portion.

2. The attachment component of claim 1, wherein the body is rotationally symmetric about the longitudinal axis.

3. The attachment component of claim 1, wherein the cavity comprises a circular or a concave cavity.

4. The attachment component of claim 1, wherein the cavity is configured to stretch and accommodate the earplug.

5. The attachment component of claim 1, wherein the unitary body is made up of stretchable material and tear resistant material.

6. The attachment component of claim 1, wherein the attachment component is formed as a single moulded component moulded by pouring stretchable and tear resistant material.

7. The attachment component of claim 1, wherein the fastening element comprises clasp comprising trigger clasp, hook-and-latch fastener, and jewelry clasp.

8. The attachment component of claim 1, wherein the fastening element comprises string, jump ring, or an accessory worn by a user.

9. The attachment component of claim 1, wherein the attachment component is coupled with earring attachments.

10. The attachment component of claim 1, wherein the attachment component is coupled with clasping necklace, glasses attachments, or an accessory worn by a user.
